SEQUENCE LISTING (4 sequences)

SEQ ID NO: 1 - DNA, 15 bp, artificial
  LNA oligomer
ccattgtcacactcc

SEQ ID NO: 2 - DNA, 8 bp, artificial
  LNA oligomer
gataagct

SEQ ID NO: 3 - DNA, 13 bp, artificial
  LNA oligomer
gtctgtggaagcg

SEQ ID NO: 4 - DNA, 12 bp, artificial
  LNA oligomer
gttgacactgtc

(19) United States
(12) Patent Application Publication
(10) Pub. No.: US 2014/0288299 A1
(43) Pub. Date: Sep. 25, 2014

(54) PROCESS

(71) Applicants: Michael Meldgaard, Hillerod (DK); Marianne Mogensen, Alsgard (DK); Christian Frauendorf, Berlin (DE); Christoph Rosenbohm, Birkerod (DK)

(72) Inventors: Michael Meldgaard, Hillerod (DK); Marianne Mogensen, Alsgard (DK); Christian Frauendorf, Berlin (DE); Christoph Rosenbohm, Birkerod (DK)

(73) Assignee: SANTARIS PHARMA A/S, Hoersholm (DK)

(21) Appl. No.: 13/811,771

(22) PCT Filed: Jul. 25, 2011
(86) PCT No.: PCT/EP11/62707

§ 371 Date: Jun. 9, 2014

Related U.S. Application Data
(60) Provisional application No. 61/367,885, filed on Jul. 27, 2010.

(30) Foreign Application Priority Data
Jul. 23, 2010 (GB) 1012418.8

(51) Int. Cl. C12N 15/10

(52) U.S. Cl. CPC C12N 15/1017
USPC 536/127

(57) ABSTRACT

The present invention relates to a process for preparing a short oligonucleotide comprising the steps of: (i) preparing a crude mixture comprising the oligonucleotide (ii) subjecting the mixture formed in step (i) to a desalting step; wherein the process does not comprise a chromatographic purification step.

The present invention relates to a process for preparing and purifying oligonucleotides, particularly very short oligonucleotide sequences. The process of the invention is ideally suited for large scale production and is significantly more cost/time effective than methods used in the art to date.

### BACKGROUND TO THE INVENTION

Recent developments in DNA/RNA technology, and in particular, antisense therapeutics, have meant that the production/purification of synthetic oligonucleotides has become of increasing importance. The purification challenges are significant and wide-ranging; on the one hand, large amounts of a few oligonucleotides must be purified to therapeutic quality (e.g. drug candidates), whereas on the other hand, large numbers of oligonucleotides must be also purified in smaller quantities for high throughput screening.

Antisense oligonucleotides are short single strands of DNA or RNA that are complementary to a chosen sequence. Most antisense drugs currently under investigation are typically about 20 nucleotides in length, but examples in the range 12-16 are also known. More recently, shorter sequences of oligonucleotides (for example, 7, 8, 9 and 10 mers) have also been found to have useful properities (see WO 2009043353; Santaris Pharma A/S). In particular, these shorter oligonucleotides have been shown to alleviate the repression of RNAs, such as mRNA, by targeting and inhibiting microRNAs in vivo.

Oligonucleotides may be prepared using solution phase or solid phase technologies. The latter technique has proved especially successful and packed-bed reactors are particularly advanced. One such example is the OligoProcess™ (Amersham Pharmacia Biotech, Inc.) which can synthesise 20 mer oligonucleotides at the 150 mmol level, producing roughly 900 g of crude material per 10 hour synthetic cycle (see Deshmukj; Large Scale Chromatographic Purification of Oligonucleotides; Handbook of Bioseparations; 2000; Vol 2, p 511-534).

Oligonucleotides are typically synthesised using phosphoramidite coupling chemistry (Sanghvi et al, 1999, Chemical synthesis and purification of phosphorothioate antisense oligonucleotides, in “Manual of Antisense Methodology” (G. Hartman and S Endres, eds), p 2-23, Kluwer Academic Publishers, NY). This is based on the original chemistry described by Beaucage and Caruthers (Tetrahedron Lett., 22, 1859, 1981). The general synthetic strategy is illustrated in FIG. 4 (reproduced from Deshmukj; Large Scale Chromatographic Purification of Oligonucleotides; Handbook of Bioseparations; 2000; Vol 2, p 511-534).

During the chemical synthesis, phosphoramidite monomers are sequentially coupled to an elongating oligonucleotide that is covalently bound to a solid support. The cycle is repeated for each nucleotide addition until the desired sequence length is achieved. The terminal 5′-DMT protecting group may be retained (“DMT-on”) or removed (“DMT-off”) depending on the subsequent purification method. The oligonucleotide is then cleaved from the solid support prior to purification, typically by treatment with ammonium hydroxide, which also serves to remove base and phosphate triester protecting groups.

There are two main purification techniques available for downstream purification, namely reverse phase (RP) purification and anion exchange (AX) chromatography. RP purification is the simpler of the two and has been widely used in large scale production and high throughput small scale applications. AX chromatography, which takes advantage of the negatively charged internucleotide linkages, may also be suitable for production scale use. For either method, the main impurities are typically truncated oligonucleotides (denoted “n−1”) that arise from failure of the coupling reaction. Other common impurities include partial phosphodiesters in which the sulfurization step to form the phosphorothioate group is incomplete.

For RP purification, the hydrophobic 5′-DMT group is generally retained on the oligonucleotide and imparts hydrophobicity to the molecule. The RP method results in excellent purity with high product recovery and is suitable for synthetic phosphodiester DNA molecules, phosphorothioate-modified oligonucleotides, synthetic RNAs, DNA-RNA chimeras and ribozymes. Silicate or organic polymer C₁₈-derivatised columns are typically used, in conjunction with weakly buffered RP eluants such as sodium or ammonium acetate mobile phases containing methanol or acetonitrile. Typically, an aqueous solution of crude DMT-on product is loaded onto the column at low mobile phase organic content. The organic content of the mobile phase is then increased to elute any DMT-off product and protecting group debris, before being stepped up a second time to elute the DMT-on material. After purification of the latter material, the DMT group is removed by acid treatment in aqueous solution. After neutralization, the salts, if excessive, are removed by precipitation (for example, using NaOAc and ethanol) and the product is lyophilized.

For AX chromatography, the hydrophobic 5′-DMT group is generally removed from the oligonucleotide whilst it is still attached to the solid support, i.e. prior to purification. In these cases, high purity oligonucleotides can be obtained using a single AX step. After the HPLC step, the oligonucleotide is desalted and lyophilized. Advantageously, purification by AX chromatography avoids the need for a post-purification detritylation step and concomitant oligomer precipitation. Moreover, AX chromatography is performed at relatively low pressure without the use of organic solvents, features that help reduce capital outlay and the cost of waste disposal. Furthermore, AX chromatography is able to resolve, at least partially, oligonucleotides that contain one phosphodiester linkage from fully thiolated oligonucleotides.

AX chromatography uses conventional anion exchange hardware typical of industrial bioseparations and the stationary phase and buffers used are suitable for production scale use. Whilst the purity of the oligonucleotides obtained is comparable with RP chromatography, the isolated yield tends to be lower, which can be addressed to some extent by recycling side fractions. Another disadvantage of AX chromatography is the requirement to desalt and concentrate the purified product, a task normally accomplished using RP HPLC or tangential flow filtration.

A comparison of the RP and AX purification techniques is shown in FIG. 5.

Other chromatographic techniques suitable for the small scale purification of oligonucleotides include hydrophobic interaction chromatography (HIC), affinity chromatography, gel permeation chromatography, mixed mode chromatography (e.g. ion-paired RP, hydroxyapetite, slalom chromatography) and the use of stationary phases that combine anion exchange and RP characteristics, such as RPC-5. In some cases, a combination of RP and AX chromatography may be used.

However, a key problem with the all of the above mentioned techniques is that they rely on a chromatographic separation step, which is both costly and time consuming, particularly if the oligonucleotides are required on a commercial scale.

The present invention therefore seeks to provide a method of purifying oligonucleotides that avoids the need for chromatography and is thus suitable for the large scale commercial manufacture of oligonucleotides.

### RELATED APPLICATIONS

This application claims priority to GB1012418.8, filed 23 Jul. 2010, and U.S. 61/367,885, filed 27 Jul. 2010. Both the priority documents are hereby incorporated by reference in their entirety.

### STATEMENT OF INVENTION

A first aspect of the invention relates to a process for preparing an oligonucleotide consisting of 6 to 25 contiguous nucleotide units, said process comprising the steps of:
(i) preparing a crude mixture comprising an oligonucleotide consisting of 6 to 25 contiguous nucleotide units;
(ii) subjecting the mixture formed in step (i) to a desalting step;
   wherein the process does not comprise a chromatographic purification step.

A second aspect of the invention relates to a process for purifying an oligonucleotide consisting of 6 to 25 contiguous nucleotide units, said process comprising subjecting the oligonucleotide to diafiltration, and wherein the process does not comprise a chromatographic purification step.

Advantageously, and in contrast to purification methods known in the art, the presently claimed process avoids the need for expensive and time consuming chromatography. Preferred aspects of the invention are set forth below and apply mutatis mutandis to both the first and second aspects of the invention.

### FIGURES

FIG. 1: Example of the purification of the oligonucleotide according to the invention.

FIG. 2: Purification as performed in example 2.

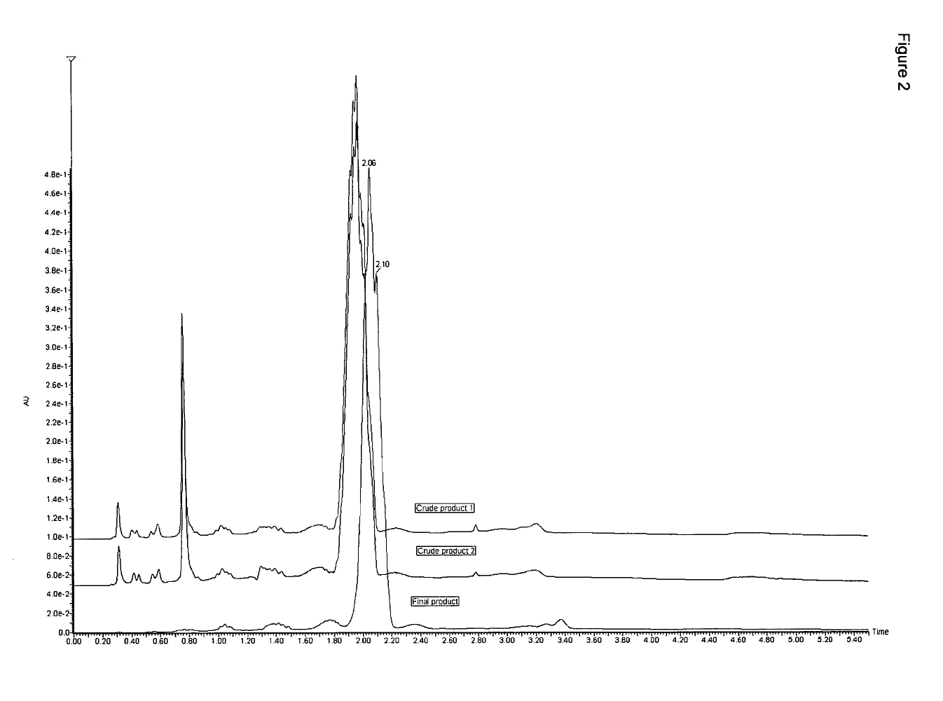

FIG. 3: Comparison between a typical prior art process and the simple desalting process of the invention.

FIG. 4: The general synthetic strategy

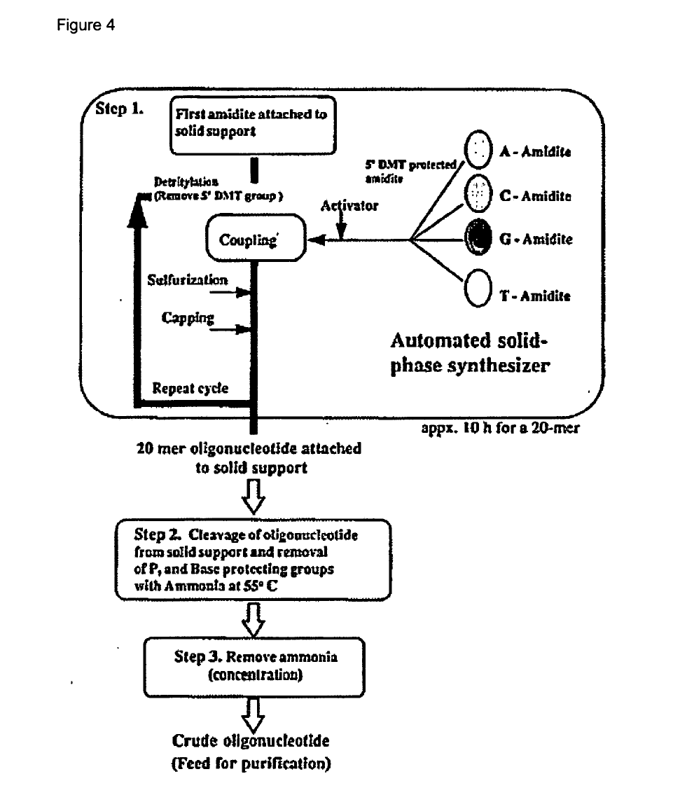

FIG. 5: A comparison of the RP and AX purification techniques

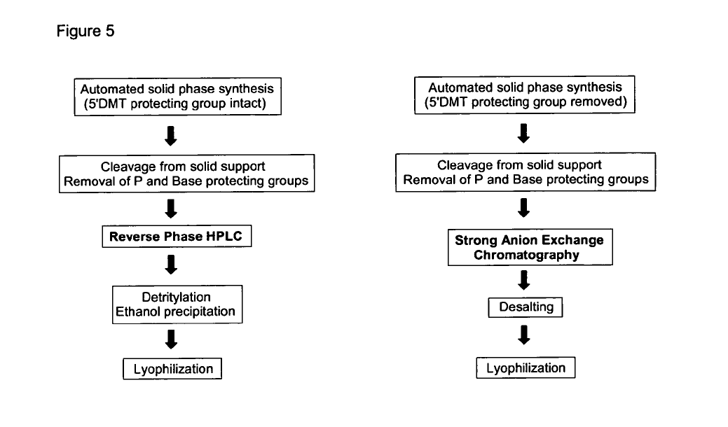

### DETAILED DESCRIPTION

As mentioned above, a first aspect of the invention relates to a process for preparing an oligonucleotide consisting of 6 to 25 contiguous nucleotide units, said process comprising the steps of:
(i) preparing a crude mixture comprising an oligonucleotide consisting of 6 to 25 contiguous nucleotide units;
(ii) subjecting the mixture formed in step (i) to a desalting step;
   wherein the process does not comprise a chromatographic purification step.

The process of the invention is centred on the surprising and unexpected observation that contrary to established practice, it is possible to prepare and purify short, and very short oligonucleotides (for example, those less than 16 or less than 12 nucleotide units in length) without the need for a chromatographic step. This opens up the possibility of preparing these oligonucleotides on a commercial scale in a much more cost effective manner to methods currently used the art. Avoiding the need for expensive and time consuming chromatographic purification has the added benefit of simplifying the overall synthetic procedure, thereby allowing for easy scale up and reduced waste. For a 2-400 mg production of oligonucleotide, the level of waste produced is typically about 5 litres of organic solvents. The method of the invention allows for a greatly reduced level of waste.

Preferably, the process of the invention does not comprise or involve the use of chromatographic purification methods such as HPLC, and in particular, RP-HPLC or AX-chromatography.

Scale of oligonucleotide synthesis: When referring to the scale of oligonucelotide synthesis we refer to the molar amount of oligonucleotide product present in the crude mixture. In some embodiments, the scale is greater than 1 μM, such as greater than 5 μM, such as greater than 10 μM, such as greater than 100 μM, such as greater than 200 μM, such as greater than 500 μM, such as greater than 1000 μM (1 mM), such as greater than 2 mM, such as greater than 5 mM, such as greater than 10 mM, such as greater than 50 mM or greater than 100 mM or greater than 200 mM. Small scale oligonucleotide synthesis is typically less than 1 μM.

Product purity: The purified oligonucleotide product obtained from the method of the invention may, in some embodiments, be at least about 75% pure, such as at least about 80% pure, such as at least about 85% pure, such as at least about 90% pure, such as at least about 95% pure. Purity of the oligonucleotide may be determined using standard assays known in the art, such as HPLC, LC-MS, or UPLC.

In some embodiments, the crude mixture formed in step (i) is prepared by the sequential coupling of phosphoroamidite monomers to a nucleotide or oligonucleotide that is covalently bound to a solid support. Preferably, the oligonucleotide is prepared by conventional methods well known in the art, for example, as described in Sanghvi et al, 1999, Chemical synthesis and purification of phosphorothioate antisense oligonucleotides, in “Manual of Antisense Methodology” (G. Hartman and S Endres, eds), p 2-23, Kluwer Academic Publishers, NY; and Beaucage and Caruthers (Tetrahedron Lett., 22, 1859, 1981).

The crude mixture is typically an unpurified product from oligonucelotide synthesis which typically comprises the oligonucleotide product as well as truncated versions of the oligonucleotide, deletion fragments as well as cleaved protection groups.

Preferably, the oligonucleotides are prepared using phosphoramidite coupling chemistry of 5′-protected nucleotides. More preferably, the 5′-protecting group is a 4,4′-dimethoxytrityl (DMT) protecting group. Other protecting groups useful in oligonucleotide synthesis are also suitable and will be familiar to the skilled person.

In one embodiment, the oligonucleotide is cleaved from the solid phase and the protecting groups are removed, such as removed using standard techniques which are well known in the art.

In another embodiment, the oligonucleotide is cleaved from the solid phase support using standard techniques and the protecting groups (e.g. DMT) are retained.

### Oligomers

The process of the present invention is suitable for purifying very short oligonucleotides, for example, those 16 nucleotide units in length or less, such as 12 nucleotide units in length or less, more preferably 6 to 12, more preferably, 7 to 10 nucleoside units in length. In some embodiments, the oligonucleotide has a length of 6, 7, 8, 9, 10, 11, 12, 13, 14, 15, 16, 17, 18, 19, 20, 21, 22, 23, 24 or 25 nucleotides in length.

Short oligonucleotides are described in more detail in WO 2009043353 (Santaris Pharma A/S), the contents of which are hereby incorporated by reference in its entirity.

The oligomers prepared by the process of the invention are single stranded oligonucleotides which optionally comprise one or more nucleotide analogues, such as LNA, which form part of, or the entire contiguous nucleotide sequence of the oligonucleotide.

The term “oligonucleotide” (or simply “oligo”), which is used interchangeably with the term “oligomer” refers, in the context of the present invention, to a molecule formed by the covalent linkage of two or more nucleotides. When used in the context of the oligonucleotide of the invention (also referred to the single stranded oligonucleotide), the term “oligonucleotide” has, for example, 7 to 10 nucleotide units, such as in individual embodiments, 7, 8, 9, or 10 nucleotide units.

As used herein, the term ‘nucleotide’ refers to nucleotides, such as DNA and RNA, as well as nucleotide analogues. In some embodiments, each nucleoside unit of the oligonucleotide is independently selected from the group consisting of LNA and DNA nucleoside units. In some embodiments, such as when the length of the oligonucleotide is between 6-12 nucleotides in length, such as between 7-10 nucleotides in length, each nucleoside unit of the oligonucleotide is a LNA nucleoside. Suitably, such LNA containing oligonucleotides may have one or more phosphorothioate linkage, including the embodiment where all internucleoisde linkages are phosphorothioate.

The nucleotide units of the oligonucleotides may be linked by phosphodiester or phosphorothioate linkages, or a mixture thereof. Preferably, the nucleotide units of the oligonucleotides are linked by phosphorothioate linkages. Alternatively, the nucleotide units of the oligonucleotides may be linked by other means, for example, by sugar linkages.

The terms “corresponding to” and “corresponds to” refer to the comparison between the nucleotide sequence of the oligomer or contiguous nucleotide sequence (a first sequence) and the equivalent contiguous nucleotide sequence of a further sequence, for example, a sub-sequence of the reverse complement of a microRNA nucleic acid target (such as the microRNA targets described in WO 2009043353), or a sequence selected from SEQ ID NO 977-1913, SEQ ID NO 1914-2850, and SEQ ID NO 2851-3787 as described in WO 2009043353. In some embodiments, the oligomer is selected from the group consisting of:

5′-^{m}Cₛ^{o}cₛAₛ^{o}tₛtₛGₛ^{o}Tₛ^{o}cₛaₛ^{m}Cₛ^{o}aₛ^{m}Cₛ^{o}tₛ^{m}Cₛ^{om}C^{o}-3′ (SEQ ID NO 1)
5′-Gₛ^{o}Aₛ^{o}Tₛ^{o}Aₛ^{o}Aₛ^{o}Gₛ^{om}Cₛ^{o}T^{o}-3′ (SEQ ID NO 2),
5′-Gₛ^{o}Tₛ^{o}cₛtₛgₛtₛgₛgₛaₛaₛGₛ^{om}Cₛ^{o}G^{o}-3′ (SEQ ID NO 3), and
5′-Gₛ^{o}Tₛ^{o}tₛgₛaₛcₛaₛcₛtₛgₛTₛ^{om}C^{o}-3′ (SEQ ID NO 4); wherein; a lowercase letter identifies a DNA unit, and an upper case letter identifies a LNA unit, ^{m}C identifies a 5-methylcytosine LNA, subscript ₛ identifies a phosphorothioate internucleoside linkage, and wherein LNA units are beta-D-oxy, as identified by a ^{o} superscript after LNA residue.

As used herein, “hybridisation” means hydrogen bonding, which may be Watson-Crick, Hoogsteen, reversed Hoogsteen hydrogen bonding, etc., between complementary nucleoside or nucleotide bases. The four nucleobases commonly found in DNA are G, A, T and C of which G pairs with C, and A pairs with T. In RNA T is replaced with uracil (U), which then pairs with A. The chemical groups in the nucleobases that participate in standard duplex formation constitute the Watson-Crick face. Hoogsteen showed a couple of years later that the purine nucleobases (G and A) in addition to their Watson-Crick face have a Hoogsteen face that can be recognised from the outside of a duplex, and used to bind pyrimidine oligonucleotides via hydrogen bonding, thereby forming a triple helix structure.

The nucleotides units each comprise a nucleobase. As used herein, the term “nucleobase” refers to nitrogenous bases including purines and pyrimidines, such as the DNA nucleobases A, C, T and G, the RNA nucleobases A, C, U and G, as well as non-DNA/RNA nucleobases, such as 5-methylcytosine (^{Me}C), isocytosine, pseudoisocytosine, 5-bromouracil, 5-propynyluracil, 5-propyny-6-fluoroluracil, 5-methylthiazoleuracil, 6-aminopurine, 2-aminopurine, inosine, 2,6-diaminopurine, 7-propyne-7-deazaadenine, 7-propyne-7-deazaguanine and 2-chloro-6-aminopurine, in particular ^{Me}C. It will be understood that the actual selection of the non-DNA/RNA nucleobase will depend on the corresponding (or matching) nucleotide present in the RNA strand which the oligonucleotide is intended to target. For example, in case the corresponding nucleotide is G it will normally be necessary to select a non-DNA/RNA nucleobase which is capable of establishing hydrogen bonds to G. In this specific case, where the corresponding nucleotide is G, a typical example of a preferred non-DNA/RNA nucleobase is ^{Me}C.

In the context of the present invention “complementary” refers to the capacity for precise pairing between two nucleotides sequences with one another. For example, if a nucleotide at a certain position of an oligonucleotide is capable of hydrogen bonding with a nucleotide at the corresponding position of a DNA or RNA molecule, then the oligonucleotide and the DNA or RNA are considered to be complementary to each other at that position. The DNA or RNA strand are considered complementary to each other when a sufficient number of nucleotides in the oligonucleotide can form hydrogen bonds with corresponding nucleotides in the target DNA or RNA to enable the formation of a stable complex. To be stable in vitro or in vivo the sequence of an oligonucleotide need not be 100% complementary to its target. The terms “complementary” and “specifically hybridisable” thus imply that the oligonucleotide binds sufficiently strong and specific to the target molecule to provide the desired interference with the normal function of the target whilst leaving the function of non-target RNAs unaffected.

In some embodiments, the oligonucleotide prepared by the process of the invention is 100% complementary to a miRNA sequence, such as a human microRNA sequence, or one of the microRNA sequences referred to in WO 2009043353.

In the context of the present invention the oligonucleotide is single stranded, this refers to the situation where the oligonucleotide is in the absence of a complementary oligonucleotide, i.e. it is not a double stranded oligonucleotide complex, such as an siRNA. It will be recognised that once purified according to the present invention and oligonucleotide may be hybridised with other oligonucleotides which may be complementary to part of or all of the oligonucleotide prepared according to the present invention, to form, for example, a siRNA.

In some embodiments, the oligonucleotide does not have a G nucleoside at the 3′ terminal position and/or the nucleoside immediately adjacent to the 3′ terminal nucleoside (i.e. at position 1 or 2 from the 3′ end).

### Gapmer Design

In some embodiments, the oligomer of the invention is a gapmer. A gapmer oligomer is an oligomer which comprises a contiguous stretch of nucleotides which is capable of recruiting an RNAse, such as RNAseH, such as a region of at least 6 or 7 DNA nucleotides, referred to herein in as region B (B), wherein region B is flanked both 5′ and 3′ by regions of affinity enhancing nucleotide analogues, such as from 1-6 nucleotide analogues 5′ and 3′ to the contiguous stretch of nucleotides which is capable of recruiting RNAse—these regions are referred to as regions A (A) and C (C) respectively.

In some embodiments, the monomers which are capable of recruiting RNAse are selected from the group consisting of DNA monomers, alpha-L-LNA monomers, C4′ alkylayted DNA monomers (see PCT/EP2009/050349 and Vester et al., Bioorg. Med. Chem. Lett. 18 (2008) 2296-2300, hereby incorporated by reference), and UNA (unlinked nucleic acid) nucleotides (see Fluiter et al., Mol. Biosyst., 2009, 10, 1039 hereby incorporated by reference). UNA is unlocked nucleic acid, typically where the C2-C3 C—C bond of the ribose has been removed, forming an unlocked “sugar” residue. Preferably the gapmer comprises a (poly)nucleotide sequence of formula (5′ to 3′), A-B-C, or optionally A-B-C-D or D-A-B-C, wherein; region A (A) (5′ region) consists or comprises of at least one nucleotide analogue, such as at least one LNA unit, such as from 1-6 nucleotide analogues, such as LNA units, and; region B (B) consists or comprises of at least five consecutive nucleotides which are capable of recruiting RNAse (when formed in a duplex with a complementary RNA molecule, such as the mRNA target), such as DNA nucleotides, and; region C (C) (3′ region) consists or comprises of at least one nucleotide analogue, such as at least one LNA unit, such as from 1-6 nucleotide analogues, such as LNA units, and; region D (D), when present consists or comprises of 1, 2 or 3 nucleotide units, such as DNA nucleotides.

In some embodiments, region A consists of 1, 2, 3, 4, 5 or 6 nucleotide analogues, such as LNA units, such as from 2-5 nucleotide analogues, such as 2-5 LNA units, such as 3 or 4 nucleotide analogues, such as 3 or 4 LNA units; and/or region C consists of 1, 2, 3, 4, 5 or 6 nucleotide analogues, such as LNA units, such as from 2-5 nucleotide analogues, such as 2-5 LNA units, such as 3 or 4 nucleotide analogues, such as 3 or 4 LNA units.

In some embodiments B consists or comprises of 5, 6, 7, 8, 9, 10, 11 or 12 consecutive nucleotides which are capable of recruiting RNAse, or from 6-10, or from 7-9, such as 8 consecutive nucleotides which are capable of recruiting RNAse. In some embodiments region B consists or comprises at least one DNA nucleotide unit, such as 1-12 DNA units, preferably from 4-12 DNA units, more preferably from 6-10 DNA units, such as from 7-10 DNA units, most preferably 8, 9 or 10 DNA units.

In some embodiments region A consist of 3 or 4 nucleotide analogues, such as LNA, region B consists of 7, 8, 9 or 10 DNA units, and region C consists of 3 or 4 nucleotide analogues, such as LNA. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 nucleotide units, such as DNA units.

Further gapmer designs are disclosed in WO2004/046160, which is hereby incorporated by reference. WO2008/113832, which claims priority from U.S. provisional application 60/977,409 hereby incorporated by reference, refers to ‘shortmer’ gapmer oligomers. In some embodiments, oligomers presented here may be such shortmer gapmers.

In some embodiments the oligomer is consisting of a contiguous nucleotide sequence of a total of 10, 11, 12, 13 or 14 nucleotide units, wherein the contiguous nucleotide sequence is of formula (5′-3′), A-B-C, or optionally A-B-C-D or D-A-B-C, wherein; A consists of 1, 2 or 3 nucleotide analogue units, such as LNA units; B consists of 7, 8 or 9 contiguous nucleotide units which are capable of recruiting RNAse when formed in a duplex with a complementary RNA molecule (such as a mRNA target); and C consists of 1, 2 or 3 nucleotide analogue units, such as LNA units. When present, D consists of a single DNA unit.

In some embodiments A consists of 1 LNA unit. In some embodiments A consists of 2 LNA units. In some embodiments A consists of 3 LNA units. In some embodiments C consists of 1 LNA unit. In some embodiments C consists of 2 LNA units. In some embodiments C consists of 3 LNA units. In some embodiments B consists of 7 nucleotide units. In some embodiments B consists of 8 nucleotide units. In some embodiments B consists of 9 nucleotide units. In certain embodiments, region B consists of 10 nucleoside monomers. In certain embodiments, region B comprises 1-10 DNA monomers. In some embodiments B comprises of from 1-9 DNA units, such as 2, 3, 4, 5, 6, 7, 8 or 9 DNA units. In some embodiments B consists of DNA units. In some embodiments B comprises of at least one LNA unit which is in the alpha-L configuration, such as 2, 3, 4, 5, 6, 7, 8 or 9 LNA units in the alpha-L-configuration. In some embodiments B comprises of at least one alpha-L-oxy LNA unit or wherein all the LNA units in the alpha-L-configuration are alpha-L-oxy LNA units. In some embodiments the number of nucleotides present in A-B-C are selected from the group consisting of (nucleotide analogue units-region B-nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In some embodiments the number of nucleotides in A-B-C are selected from the group consisting of: 2-7-1, 1-7-2, 2-7-2, 3-7-3, 2-7-3, 3-7-2, 3-7-4, and 4-7-3. In certain embodiments, each of regions A and C consists of three LNA monomers, and region B consists of 8 or 9 or 10 nucleoside monomers, preferably DNA monomers. In some embodiments both A and C consists of two LNA units each, and B consists of 8 or 9 nucleotide units, preferably DNA units. In various embodiments, other gapmer designs include those where regions A and/or C consists of 3, 4, 5 or 6 nucleoside analogues, such as monomers containing a 2′-O-methoxyethyl-ribose sugar (2′-MOE) or monomers containing a 2′-fluoro-deoxyribose sugar, and region B consists of 8, 9, 10, 11 or 12 nucleosides, such as DNA monomers, where regions A-B-C have 3-9-3, 3-10-3, 5-10-5 or 4-12-4 monomers. Further gapmer designs are disclosed in WO 2007/146511A2, hereby incorporated by reference.

### Length

When referring to the length of a nucleotide molecule as referred to herein, the length corresponds to the number of monomer units, i.e. nucleotides, irrespective as to whether those monomer units are nucleotides or nucleotide analogues. With respect to nucleotides, the terms monomer and unit are used interchangeably herein.

The process of the present invention is particularly suitable for the purification of short oligonucleotides, for example, consisting of 6 to 16 nucleotides, or 6 to 12 nucleotides, such as 7 to 10 nucleotides, for example, 7, 8, 9 or 10 nucleotides, or 7 to 9 nucleotides.

### Nucleotide Analogues

In some embodiments of the invention, the oligonucleotides prepared by the process of the invention comprise at least one nucleotide analogue, for example, a Locked Nucleic Acid (LNA).

The process of the present invention is particularly suitable for purifying short oligonucleotides of 6 to 16, such as 6 to 12 nucleotides, such as, 7, 8, 9, 10 nucleotides, such as 7, 8 or 9 nucleotides, wherein at least 50%, such as 55%, 60%, 65%, 70%, 75%, 80%, 85%, 90%, 95% or such as 100% of the nucleotide units of the oligomer are (preferably high affinity) nucleotide analogues, such as a Locked Nucleic Acid (LNA) nucleotide unit.

In some embodiments, the oligonucleotide is 7, 8 or 9 nucleotides long, and comprises a contiguous nucleotide sequence which is complementary to a seed region of a human or viral microRNA, and wherein at least 75%, preferably at least 80%, preferably at least 85%, preferably at least 90%, preferably at least 95%, or 100% of the nucleotides are Locked Nucleic Acid (LNA) nucleotide units.

In such oligomers, in some embodiments, the linkage groups are other than phosphodiester linkages. Preferably, the linkage groups are phosphorothioate linkages.

In some embodiments, all of the nucleotide units of the contiguous nucleotide sequence are LNA nucleotide units. In a further preferred embodiment, all of the nucleotides of the oligomer are LNA and all of the internucleoside linkage groups are phosphothioate.

In some embodiments, the contiguous nucleotide sequence consists of 7 nucleotide analogues. In another preferred embodiment, the contiguous nucleotide sequence consists of 8 nucleotide analogues. In another preferred embodiment, the contiguous nucleotide sequence consists of 9 nucleotide analogues.

In some embodiments the oligomer comprises at least one LNA monomer, for example, such as 2, 3, 4, 5, 6, 7, 8, 9 or 10 LNA monomers. As described below, the contiguous nucleotide sequence may consist only of LNA units (including linkage groups, such as phosphorothioate linkages), or may consists of LNA and DNA units, or LNA and other nucleotide analogues. In some embodiments, the contiguous nucleotide sequence comprises either one or two DNA nucleotides, the remainder of the nucleotides being nucleotide analogues, such as LNA units.

In some embodiments, the contiguous nucleotide sequence consists of 6 nucleotide analogues and a single DNA nucleotide. In some embodiments embodiment, the contiguous nucleotide consists of 7 nucleotide analogues and a single DNA nucleotide. In some embodiments, the contiguous nucleotide sequence consists of 8 nucleotide analogues and a single DNA nucleotide. In some embodiments, the contiguous nucleotide sequence consists of 9 nucleotide analogues and a single DNA nucleotide. In some embodiments, the contiguous nucleotide sequence consists of 7 nucleotide analogues and two DNA nucleotides. In some embodiments, the contiguous nucleotide sequence consists of 8 nucleotide analogues and two DNA nucleotides.

In some embodiments, the contiguous nucleotide sequence comprises or consists of 7, 8, 9 or 10, preferably contiguous, LNA nucleotide units.

In some embodiments, the oligonucleotide of the invention is 7, 8 or 9 nucleotides long, and comprises a contiguous nucleotide sequence which is complementary to a seed region of a human or viral microRNA, and wherein at least 80% of the nucleotides are LNA, and wherein at least 80% (for example, such as 85%, 90%, 95%, or 100%) of the internucleotide bonds are phosphorothioate bonds. It will be recognised that the contiguous nucleotide sequence of the oligomer (a seedmer) may extend beyond the seed region.

In some embodiments, the oligonucleotide of the invention is 7 nucleotides long, wherein all of the nucleotides are LNA.

In some embodiments, the oligonucleotide of the invention is 8 nucleotides long, of which up to 1 nucleotide may be other than LNA. In some embodiments, the oligonucleotide of the invention is 9 nucleotides long, of which up to 1 or 2 nucleotides may be other than LNA. In some embodiments, the oligonucleotide of the invention is 10 nucleotides long, of which 1, 2 or 3 nucleotides may be other than LNA. The nucleotides ‘other than LNA, may for example, be DNA, or a 2’ substituted nucleotide analogues.

High affinity nucleotide analogues are nucleotide analogues which result in oligonucleotides having a higher thermal duplex stability with a complementary RNA nucleotide than the binding affinity of an equivalent DNA nucleotide. This may be determined by measuring the melting temperature of the duplex (Tₘ).

In some embodiments, the nucleotide analogue units present in the contiguous nucleotide sequence are each independently selected from the group consisting of 2′-O-alkyl-RNA unit, 2′-OMe-RNA unit, 2′-amino-DNA unit, 2′-fluoro-DNA unit, LNA unit, PNA unit, HNA unit, INA unit, and a 2′MOE RNA unit.

In some embodiments, the nucleotide analogue units present in the contiguous nucleotide sequence are each independently selected from the group consisting of 2′-O-alkyl-RNA unit, 2′-OMe-RNA unit, 2′-amino-DNA unit, 2′-fluoro-DNA unit, LNA unit, and a 2′MOE RNA unit.

The term 2′fluoro-DNA refers to a DNA analogue with a substitution to fluorine at the 2′ position (2′F). 2′fluoro-DNA is a preferred form of 2′fluoro-nucleotide. 2′-deoxy-2′-fluoro-arabinonucleic acid (FANA) is another example.

In some embodiments, the oligomer comprises at least 4 nucleotide analogue units, such as at least 5 nucleotide analogue units, such as at least 6 nucleotide analogue units, such as at least 7 nucleotide analogue units, such as at least 8 nucleotide analogue units, such as at least 9 nucleotide analogue units, such as 10, nucleotide analogue units.

In some embodiments, the oligomer comprises at least 3 LNA units, such as at least 4 LNA units, such as at least 5 LNA units, such as at least 6 LNA units, such as at least 7 LNA units, such as at least 8 LNA units, such as at least 9 LNA units, such as 10 LNA units.

In some embodiments, at least one of the nucleobases in the oligonucleotide is cytosine or guanine, such as from 1 to 10 of the nucleobases, more specifically, 2, 3, 4, 5, 6, 7, 8, or 9 of the nucleobases.

In some embodiments, at least two of the nucleobases in the oligonucleotide are selected from cytosine and guanine. In some embodiments at least three of the nucleobases in the oligonucleotide are selected from cytosine and guanine. In some embodiments, at least four of the nucleobases in the oligonucleotide are selected from cytosine and guanine. In some embodiments, at least five of the nucleobases in the oligonucleotide are selected from cytosine and guanine. In some embodiments, at least six of the nucleobases in the oligonucleotide are selected from cytosine and guanine. In some embodiments, at least seven of the nucleobases in the oligonucleotide are selected from cytosine and guanine. In some embodiments, at least eight of the nucleobases in the oligonucleotide are selected from cytosine and guanine.

Whilst it is envisaged that other nucleotide analogues, such as 2′-MOE RNA or 2′-fluoro nucleotides may be useful in the oligomers according to the invention, it is preferred that the oligomers have a high proportion, such as at least 50%, of LNA nucleotides.

The nucleotide analogue may be a DNA analogue such as a DNA analogue where the 2′—H group is substituted with a substitution other than —OH (RNA) e.g. by substitution with —O—CH₃, —O—CH₂—CH₂—O—CH₃, —O—CH₂—CH₂—CH₂—NH₂, —O—CH₂—CH₂—CH₂—OH or —F. The nucleotide analogue may be RNA analogues such as those which have been modified in their 2′—OH group, e.g. by substitution with a group other than —H (DNA), for example —O—CH₃, —O—CH₂—CH₂—O—CH₃, —O—CH₂—CH₂—CH₂—NH₂, —O—CH₂—CH₂—CH₂—OH or —F.

### LNA

When used in the present context, the terms “LNA unit”, “LNA monomer”, “LNA residue”, “locked nucleic acid unit”, “locked nucleic acid monomer” or “locked nucleic acid residue”, refer to a bicyclic nucleoside analogue. LNA units are described in inter alia WO 99/14226, WO 00/56746, WO 00/56748, WO 01/25248, WO 02/28875, WO 03/006475 and WO 03/095467. The LNA unit may also be defined with respect to its chemical formula. Thus, an “LNA unit”, as used herein, has the chemical structure shown in Scheme 3 below:

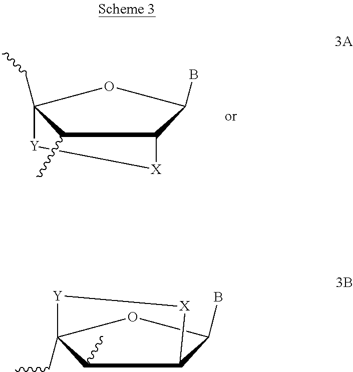

wherein X is selected from the group consisting of O, S and NR^{H}, where R^{H} is H or C₁₋₄-alkyl; Y is (—CH₂)ᵣ, where r is an integer of 1-4; and B is a nitrogenous base. In some embodiments of the invention, r is 1 or 2 (r=2 is ENA), in particular 1, i.e. preferred LNA units have the chemical structures shown in Scheme 4 below:

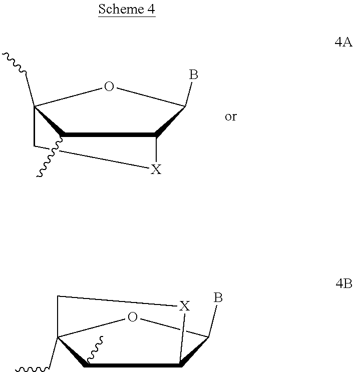

wherein X and B are as defined above.

In some embodiments, the LNA units incorporated in the oligonucleotides of the invention are independently selected from the group consisting of thio-LNA units, amino-LNA units and oxy-LNA units.

Thus, the thio-LNA units preferably have the chemical structures shown in Scheme 5 below:

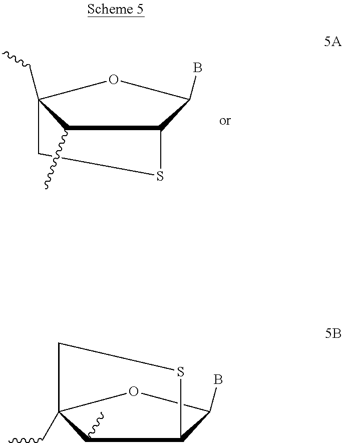

wherein B is as defined above.

Preferably, the thio-LNA unit is in its beta-D-form, i.e. having the structure shown in 5A above.

Likewise, the amino-LNA units preferably have the chemical structures shown in Scheme 6 below:

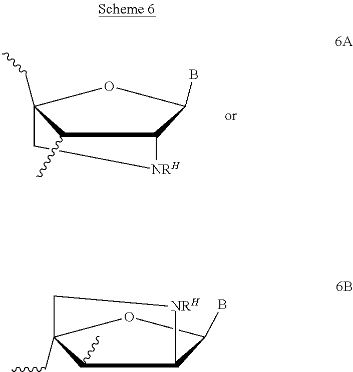

wherein B and R^{H} are as defined above.

Preferably, the amino-LNA unit is in its beta-D-form, i.e. having the structure shown in 6A above.

The oxy-LNA units preferably have the chemical structures shown in Scheme 7 below:

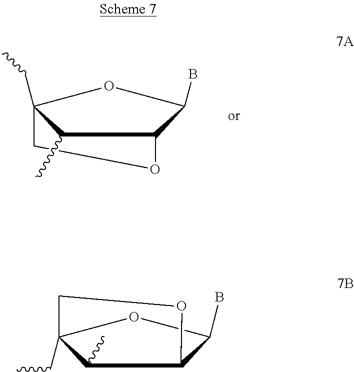

wherein B is as defined above.

Preferably, the oxy-LNA unit is in its beta-D-form, i.e. having the structure shown in 5A above.

As indicated above, B is a nitrogenous base which may be of natural or non-natural origin. Specific examples of nitrogenous bases include adenine (A), cytosine (C), 5-methylcytosine (^{Me}C), isocytosine, pseudoisocytosine, guanine (G), thymine (T), uracil (U), 5-bromouracil, 5-propynyluracil, 5-propyny-6, 5-methylthiazoleuracil, 6-aminopurine, 2-aminopurine, inosine, 2,6-diaminopurine, 7-propyne-7-deazaadenine, 7-propyne-7-deazaguanine and 2-chloro-6-aminopurine.

The term “thio-LNA unit” refers to an LNA unit in which X in Scheme 3 is S. A thio-LNA unit can be in both the beta-D form and in the alpha-L form. Generally, the beta-D form of the thio-LNA unit is preferred. The beta-D-form and alpha-L-form of a thio-LNA unit are shown in Scheme 5 as compounds 5A and 5B, respectively.

The term “amino-LNA unit” refers to an LNA unit in which X in Scheme 3 is NH or NR^{H}, where R^{H} is hydrogen or C₁₋₄-alkyl. An amino-LNA unit can be in both the beta-D form and in the alpha-L form. Generally, the beta-D form of the amino-LNA unit is preferred. The beta-D-form and alpha-L-form of an amino-LNA unit are shown in Scheme 6 as compounds 6A and 6B, respectively.

The term “oxy-LNA unit” refers to an LNA unit in which X in Scheme 3 is O. An Oxy-LNA unit can be in both the beta-D form and in the alpha-L form. Generally, the beta-D form of the oxy-LNA unit is preferred. The beta-D form and the alpha-L form of an oxy-LNA unit are shown in Scheme 7 as compounds 7A and 7B, respectively.

In the present context, the term “C₁₋₆-alkyl” is intended to mean a linear or branched saturated hydrocarbon chain wherein the chain has from one to six carbon atoms, such as methyl, ethyl, n-propyl, isopropyl, n-butyl, isobutyl, sec-butyl, tert-butyl, pentyl, isopentyl, neopentyl and hexyl. A branched hydrocarbon chain is intended to mean a C₁₋₆-alkyl substituted at any carbon with a hydrocarbon chain.

In the present context, the term “C₁₋₄-alkyl” is intended to mean a linear or branched saturated hydrocarbon chain wherein the chain has from one to four carbon atoms, such as methyl, ethyl, n-propyl, isopropyl, n-butyl, isobutyl, sec-butyl and tert-butyl. A branched hydrocarbon chain is intended to mean a C₁₋₄-alkyl substituted at any carbon with a hydrocarbon chain.

As used herein the term “C₁₋₆-alkoxy” is intended to mean C₁₋₆-alkyl-oxy, such as methoxy, ethoxy, n-propoxy, isopropoxy, n-butoxy, isobutoxy, sec-butoxy, tert-butoxy, pentoxy, isopentoxy, neopentoxy and hexoxy.

In the present context, the term “C₂₋₆-alkenyl” is intended to mean a linear or branched hydrocarbon group having from two to six carbon atoms and containing one or more double bonds. Illustrative examples of C₂₋₆-alkenyl groups include allyl, homo-allyl, vinyl, crotyl, butenyl, butadienyl, pentenyl, pentadienyl, hexenyl and hexadienyl. The position of the unsaturation (the double bond) may be at any position in the group. In the present context the term “C₂₋₆-alkynyl” is intended to mean a linear or branched hydrocarbon group containing from two to six carbon atoms and containing one or more triple bonds. Illustrative examples of C₂₋₆-alkynyl groups include acetylene, propynyl, butynyl, pentynyl and hexynyl. The position of unsaturation (the triple bond) may be at any position in the group. More than one bond may be unsaturated such that the “C₂₋₆-alkynyl” is a di-yne or enedi-yne as is known to the person skilled in the art.

When referring to substituting a DNA unit by its corresponding LNA unit in the context of the present invention, the term “corresponding LNA unit” is intended to mean that the DNA unit has been replaced by an LNA unit containing the same nitrogenous base as the DNA unit that it has replaced, e.g. the corresponding LNA unit of a DNA unit containing the nitrogenous base A also contains the nitrogenous base A. The exception is that when a DNA unit contains the base C, the corresponding LNA unit may contain the base C or the base ^{Me}C, preferably ^{Me}C.

As used herein, the term “non-LNA unit” refers to a nucleoside different from an LNA-unit, i.e. the term “non-LNA unit” includes a DNA unit as well as an RNA unit. A preferred non-LNA unit is a DNA unit.

The terms “unit”, “residue” and “monomer” are used interchangeably herein.

The term “at least one” encompasses an integer larger than or equal to 1, such as 1, 2, 3, 4, 5, 6, 7, 8, 9, 10, 11, 12, 13, 14, 15, 16, 17, 18, 19, 20 and so forth.

The terms “a” and “an” as used about a nucleotide, an agent, an LNA unit, etc., is intended to mean one or more. In particular, the expression “a component (such as a nucleotide, an agent, an LNA unit, or the like) selected from the group consisting of . . . ” is intended to mean that one or more of the cited components may be selected. Thus, expressions like “a component selected from the group consisting of A, B and C” is intended to include all combinations of A, B and C, i.e. A, B, C, A+B, A+C, B+C and A+B+C.

### Internucleoside Linkages

The term “internucleoside linkage group” is intended to mean a group capable of covalently coupling together two nucleotides, such as between DNA units, between DNA units and nucleotide analogues, between two non-LNA units, between a non-LNA unit and an LNA unit, and between two LNA units, etc. Examples include phosphate, phosphodiester groups and phosphorothioate groups.

In some embodiments, at least one of the internucleoside linkages in the oligomer is a phosphodiester linkage. However, phosphorothioate linkages are particularly preferred.

Typical internucleoside linkage groups in oligonucleotides are phosphate groups, but these may be replaced by internucleoside linkage groups differing from phosphate. In a further preferred embodiment of the invention, the oligonucleotide of the invention is modified in its internucleoside linkage group structure, i.e. the modified oligonucleotide comprises an internucleoside linkage group which differs from phosphate. Accordingly, in a preferred embodiment, the oligonucleotide according to the present invention comprises at least one internucleoside linkage group which differs from phosphate. Specific examples of internucleoside linkage groups include (—O—P(O)₂—O—), —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, —NR^{H}—CO—NR^{H}—, —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}—CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, where R^{H} is hydrogen or C₁₋₄-alkyl.

When the internucleoside linkage group is modified, the internucleoside linkage group is preferably a phosphorothioate group (—O—P(O,S)—O—). In a preferred embodiment, all internucleoside linkage groups of the oligonucleotides according to the present invention are phosphorothioate.

In some embodiments, the internucleoside linkages are sulphur (S) containing linkages. The internucleoside linkages may be independently selected, or all be the same, such as phosphorothioate linkages.

In one embodiment, at least 75%, preferably at least 80% or 85% or 90% or 95% or all of the internucleoside linkages present between the nucleotide units of the contiguous nucleotide sequence are phosphorothioate internucleoside linkages.

### Oligomer Design

In some embodiments, the first nucleotide of the oligomer, counting from the 3′ end, is a nucleotide analogue, such as an LNA unit. In one embodiment, which may be the same or different, the last nucleotide of the oligomer, counting from the 3′ end, is a nucleotide analogue, such as an LNA unit.

In some embodiments, the second nucleotide of the oligomer, counting from the 3′ end, is a nucleotide analogue, such as an LNA unit.

In some embodiments, the ninth and/or the tenth nucleotide of the oligomer, counting from the 3′ end, is a nucleotide analogue, such as an LNA unit.

In some embodiments, the ninth nucleotide of the oligomer, counting from the 3′ end is a nucleotide analogue, such as an LNA unit.

In some embodiments, the tenth nucleotide of the oligomer, counting from the 3′ end is a nucleotide analogue, such as an LNA unit.

In some embodiments, both the ninth and the tenth nucleotide of the oligomer, calculated from the 3′ end are nucleotide analogues, such as LNA units.

In some embodiments, the oligomer does not comprise a region of more than 3 consecutive DNA nucleotide units. In some embodiments, the oligomer according to the invention does not comprise a region of more than 2 consecutive DNA nucleotide units.

In another preferred embodiment, the oligomer comprises a region consisting of at least two consecutive nucleotide analogue units, such as at least two consecutive LNA units.

In another preferred embodiment, the oligomer comprises a region consisting of at least three consecutive nucleotide analogue units, such as at least three consecutive LNA units.

### Synthesis of the Oligomers

The oligonucleotides described herein may be prepared using standard solid phase oligonucleotide synthesis. Suitable methodology will be familiar to the skilled artisan (see, for example, Sanghvi et al, 1999, Chemical synthesis and purification of phosphorothioate antisense oligonucleotides, in “Manual of Antisense Methodology” (G. Hartman and S Endres, eds), p 2-23, Kluwer Academic Publishers, NY; Deshmukj; Large Scale Chromatographic Purification of Oligonucleotides; Handbook of Bioseparations; 2000; Vol 2, p 511-534; Capaldi, D. C., Scozzari, A. N., Manufacturing and Analytical Processes for 2′-O-(2-Methoxyethyl)-Modified Oligonucleotides, in Antisense Drug Technology, 2.ed., Crooke, S. T., ed, CRC Press, 2008, Chapter 14, p 401-434). By way of example, the oligonucleotide may be prepared using a solid phase synthesizer such as, for example, an ABI-type bench synthesizer, a Millipore 8800 DNA synthesizer or a GE Oligopilor or OligoProcess synthesizer. The scale of oligonucelotide synthesis may be varied by selection of the appropriate oligonucleotide synthesizer, for example for 100 mmol scale synthesis an Oligo Process (GE Healthcare) may be used.

### Purification of Oligomers

The present invention involves purifying oligonucleotides without the need for chromatography. Crude oligonucleotides are prepared by conventional methods such as the solid phase techniques described above. The crude oligonucleotide is then cleaved from the solid phase support. Typically, the solid support is removed by filtration and the resulting solution is lyophilized.

In some embodiments, the crude oligonucleotide is cleaved from the solid phase support by treatment with aqueous ammonium hydroxide which also serves to remove base and phosphate triester protecting groups. Thus, in some embodiments, the crude oligonucleotide is DMT-off.

In another embodiment, the crude oligonucleotide is DMT-on.

The oligonucleotide is then subjected to a desalting step.

As used herein, the term “desalting” refers to a process by which impurities, such as inorganic salts, are removed from a mixture. When using a dried (lyophilized) crude oligonucleotide, as part of an initial step of the desalting process, the oligonucleotide is either dissolved in an electrolyte solution, or is dissolved in a suitable solvent, and an electrolyte is subsequently added to the oligonucleotide solution. Water is typically used as the solvent. The electrolyte may, for example, be a metal salt, such as a sodium or potassium salt, such as a sodium or potassium halogen salt, such as KCl, NaCl, or NaBr. The metal salt acts as a counter ion for the oligonucleotide anion. In some embodiments, the pH of the oligonucleotide solution may be adjusted to a pH of 7 or above, such as a pH of between about 7 to about pH 8. The suitable pH of the oligonucelotide solution may be achieved by using a basic solvent to dissolve the oligonucleotide, or, as is detailed below, by adjusting the pH of the oligonucleotide solution to a pH of 7 or higher.

In some embodiments, the crude oligonucleotide is dissolved in metal salt, such as a saline (NaCl), solution (such as 0.9% NaCl).

Preferably, the pH of the oligonucleotide solution is adjusted with a base, for example, aqueous sodium hydroxide solution.

Preferably, the pH is adjusted to about 7 to about 8. Preferably, the pH is adjusted by the addition of an aqueous solution of sodium hydroxide (for example, using a 10 mM NaOH solution).

Preferably, the oligonucleotide solution is then subjected to diafiltration.

The diafiltration may be carried out using commercially available instruments such as Crossflow (GE Healthcare) and Cogent M (Millipore). Other suitable commercially available instruments will be familiar to the skilled artisan.

As used herein, the term “desalting” which is used interchangeably with the term “diafiltration” refers to a membrane based separation technique that is used to reduce, remove or exchange salts and other small molecule contaminants from a sample. The technique is based on the fact that salts and other small molecule contaminants (the “permeating species”) can pass through the membrane, whereas the oligonucleotide molecules are too large to pass through.

In some embodiments, the oligonucleotide is purified using continuous diafiltration, i.e. a solution of the oligonucleotide is continuously recycled through a membrane filtration device so that the process stream containing the permeating species is removed. New solvent (i.e. “clean” liquid) is added to the reaction vessel while the permeating material is being removed. The new solvent is added at the same rate as the permeate flow (known as “constant volume wash procedure”), thereby causing the reactor contents to be free of membrane-permeating species within a brief period of time.

In some embodiments, the oligonucleotide is purified using batch diafiltration. Typically, the oligonucleotide solution is diluted by a factor of two using “clean” liquid, brought back to the original concentration by filtration, and the whole process repeated several times to achieve the required concentration contaminant.

In some embodiments, the oligonucleotide solution is subjected to diafiltration using a closed circuit, i.e. the flow goes from a reservoir containing the oligonucleotide solution through a pump to the filter, to a detector and back to the reservoir. Preferably, the detector is a UV detector or a conductivity detector or a combination thereof.

In some embodiments, the closed circuit further comprises a pressure regulator, preferably at the exit from the filter to allow the pressure across the membrane to be adjusted during the diafiltration.

The pump may be any conventional pump, for example, an HPLC pump.

The membrane used in the diafiltration step may, for example, be a commercially available membrane, such as, for example, a Pellicon 2 “mini” filter (Millipore).

In some embodiments, the membrane has a cutoff of in the range of from about 500 to about 5000, or about 500 to about 3000, such as in the range of from about 800 to about 2000, or at least 1000 Da. In some embodiments the membrane has a cutoff of about 1000 Da.

Preferably, the flow rate is from about 50 to about 2000 ml/min, such as from about 50 to about 1000 ml/min, such as from about 50 to about 500 ml/min, more preferably from about 200 to about 400 ml/min. In some embodiments, the flow rate is about 300 ml/min.

Preferably, the pressure over the membrane is adjusted to between about 1 to about 3.5 bar, such as between about 2 to about 3 bar. In some embodiments, the pressure over the membrane is kept at about 2.5 Bar.

Typically, the sample is loaded into the apparatus by pouring into the reservoir. As the desalting progresses, a flow of solvent goes from the filter to the waste and the sample is concentrated, is preferably monitored by a UV detector. In some embodiments, the solvent is water, such as water that has been purified and deionized to a high degree by a water purification system (e.g. purified or pure water), such as, for example, MilliQ water (Millipore).

When the sample has been loaded, the solvent is added stepwise until a uniform conductivity level over two or more solvent additions has been reached.

In some embodiments, the oligonucleotide solution is subjected to diafiltration for a time period of from about 30 to about 300 minutes, such as from about 60 to about 200 minutes.

In some aspects, when the sample has been desalted, the flow is stopped and the flow path is changed from filter to reservoir to filter to a suitable receptacle. The pressure over the membrane is released and the pump restarted. Preferably, the reservoir is washed until the UV detector signal reaches the baseline.

The desalted sample may, optionally, then be frozen (for example, by placing in a dry ice acetone bath) and subjected to lyophilization.

The present invention is further illustrated by way of the following non-limiting examples, and with reference to the following figures, wherein:

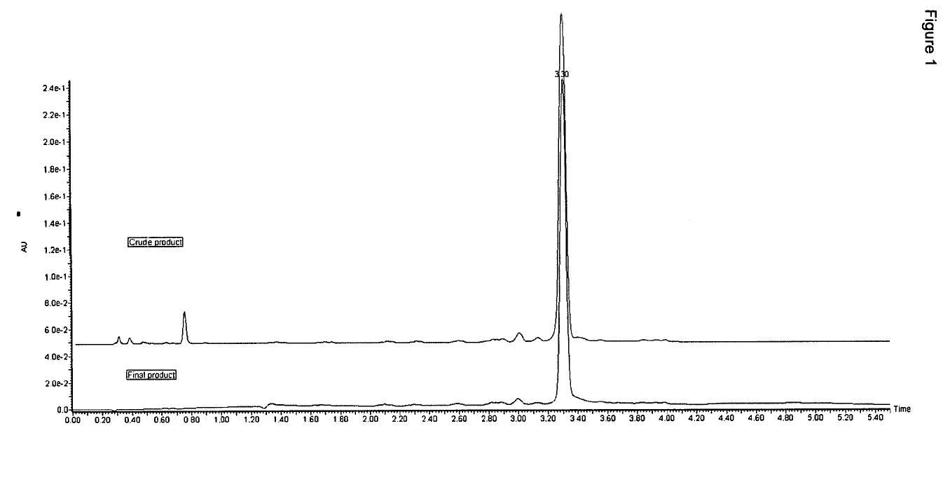

FIG. 1 shows a UPLC chromatograms of two different crude batches (1 mmol synthesis batches) prepared in accordance with Example 2, and the chromatogram of the final product. The chromatograms clearly show that significant amounts of impurities are removed during the process of the invention.

Some of the advantages of the process of the invention are illustrated in the following examples, and summarized in the table below:

| | 8-mer LNA | | | | 15-mer LNA-DNA gapmer | | | |
|---|---|---|---|---|---|---|---|---|
| | Old | | New | | Old | | New | |
| | process | | process | | process | | process | |
| Yield | 0.77 | g | 3.2 | g | 0.68 | g | 1.5 | g |
| Purity | 95.5% | | 96.3% | | 96.8% | | 89.6% | |
| Waste generation * | ++++ | | ++ | | ++++ | | ++ | |
| Time | ++++ | | + | | ++++ | | + | |
| Synthesis Scale | 0.6 | mmol | 2 | mmol | 0.26 | mmol | 0.52 | mmol |
| Yield (g/mmol) | 1.3 | g/mmol | 1.6 | g/mmol | 2.6 | g/mmol | 2.9 | g/mmol |

* waste generated during HPLC purification and desalting

### EXAMPLES

### Example 1

### Purification by desalting

An 8-mer LNA oligonucleotide was synthesized in a 100 μmol synthesis scale, cleaved and deprotected using standard procedures to give a solution of the oligonucleotide in aqueous ammonium hydroxide. The solid support was removed by filtration and the solution was lyophilized. The lyophilized oligonucleotide (DMT-off; 250 mg) was dissolved in saline (0.9% NaCl, 500 ml) and pH was adjusted to 7-8 with an aqueous solution of NaOH (10 mM).

To purify the oligonucleotide by diafiltration a CrossFlow instrument (GE Healthcare) equipped with a Pellicon 2 “mini” filter having a cutoff at 1000 Da (Millipore) was used. Part of the sample was loaded onto the CrossFlow (350 ml) and a flow parallel to the membrane surface without activating the permeate pump was started. When the desired flowrate (300 ml/min) was reached, the permeate pump was activated and the flowrate of the permeate flow was constantly adjusted to keep a trans membrane pressure (TMP) of approximately 2.5 Bar. Sample was continuously loaded on the crossflow at the same rate as permeate was removed. Once the loading of oligonucleotide sample was completed, the sample volume was reduced to 200 ml by stopping inlet and keeping the permeate pump running and the diafiltration was then continued at constant retenate volume by adding Milli Q water at the same rate as permeate was withdrawn.

The process was continued until a low and steady conductivity of the permeate was achieved (σ<0.7 mS/cm and Δσ<0.2 mS/cm min) and then, the flow of MilliQ water was replaced with a flow of WFI water. The product was eluted from the system which subsequently was flushed with WFI water. The product was then removed from the system and lyophilized to give the final product (180 mg, 62 μmol).

### Example 2

An 8-LNA was synthesized in 2×1 mmol scale, cleaved and deprotected using standard procedures. After removal of the solid support and lyophilization the oligonucleotide was dissolved in MilliQ water (800 ml), a solution of NaCl (2M in 10 mM NaOH, 100 ml) and pH was finally adjusted to 8 using an aqueous solution of NaOH (2M).

To purify the oligonucleotide by diafiltration a CrossFlow instrument (GE Healthcare) equipped with a Pellicon 2 “mini” filter having a cutoff at 1000 Da (Millipore) was used. Part of the sample was loaded onto the CrossFlow (350 ml) and a flow parallel to the membrane surface without activating the permeate pump was started. When the desired flowrate (300 ml/min) was reached, the permeate pump was activated and the flowrate of the permeate flow was constantly adjusted to keep a TMP of approximately 2.5 Bar. Sample was continuously loaded on the crossflow at the same rate as permeate was removed. Once the loading of oligonucleotide sample was completed, the sample volume was reduced to 200 ml by stopping inlet and keeping the permeate pump running and the diafiltration was then continued at constant retenate volume by adding Milli Q water at the same rate as permeate was withdrawn.

The process was continued until a low and steady conductivity of the permeate was achieved (σ<0.7 mS/cm and Δσ<0.2 mS/cm min) and then, the flow of MilliQ water was replaced with a flow of WFI water. The product was eluted from the system which subsequently was flushed with WFI water. The product was then removed from the system and lyophilized to give the final product

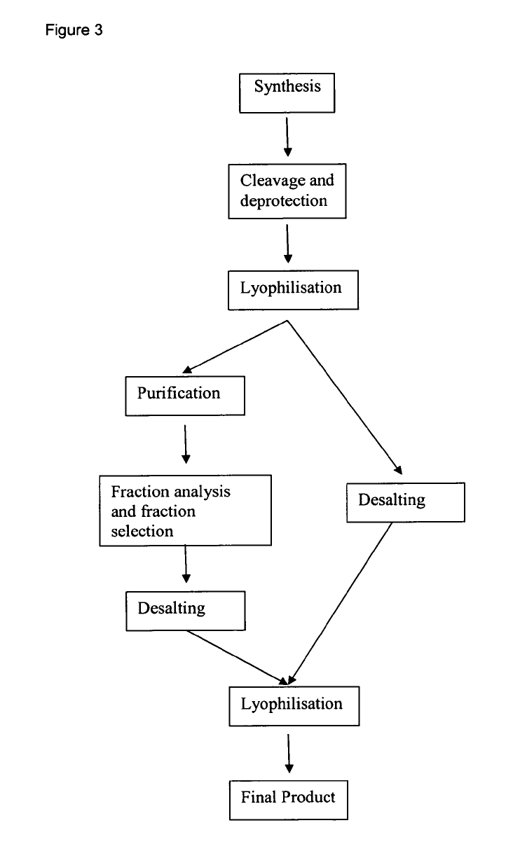

Additional syntheses (4×1 mmol and 2×2 mmol) of the same compound were produced and purified in the same manner. All 4 purification runs were pooled to give 14.5 g (5 mmol) of material of a high quality (FIG. 3).

### Example 3

A 16-mer LNA-DNA gap-mer was synthesized in 100 μmole synthesis scale using standard procedures, cleaved and deprotected using standard procedures to give a solution of the oligonucleotide in aqueous ammonium hydroxide. The solid support was removed by filtration and the solution was lyophilized. The lyophilized oligonucleotide (440 mg) was dissolved and purified by desalting as described in example 1 and lyophilized to give the final product (350 mg, 67 μmol).

### Example 4

A 14-mer LNA-DNA gap-mer was synthesized in 1 mmole synthesis scale using standard synthesis procedures. The oligonucleotide was cleaved and deprotected using standard procedures to give a solution of the oligonucleotide in aqueous ammonium hydroxide. The solid support was removed by filtration and the solution was lyophilized. The lyophilized oligonucleotide (4.3 g) was dissolved in a solution of NaCl (2M in 10 mM NaOH, 150 ml), water (650 ml) was added and the pH was adjusted to 7.7 with an aqueous solution of NaOH (1M). The oligonucleotide containing solution was purified by desalting as described in the previous examples and lyophilized to give the final product (2.6 g, 0.56 mmol).

### Example 5

An 8-mer LNA oligonucleotide was synthesized in 2 mmol synthesis scale using standard synthesis procedures. The oligonucleotide was cleaved and deprotected using standard procedures to give a solution of the oligonucleotide in aqueous ammonium hydroxide. The solid support was removed by filtration and the solution was lyophilized. The lyophilized oligonucleotide (4.4 g) was dissolved in a solution of NaCl (2M in 10 mM NaOH, 400 ml), water (600 ml) was added and the pH was adjusted to 7-8 with an aqueous solution of NaOH (1M). The oligonucleotide containing solution was purified by desalting as described in the previous examples and lyophilized to give the final product (3.1 g, 1.1 mmol).

### Example 6

A 13-mer LNA oligoenucleotide was synthesized in 200 μmol synthesis scale using standard synthesis procedures. The oligonucleotide was cleaved and deprotected using standard procedures to give a solution of the oligonucleotide in aqueous ammonium hydroxide. The solid support was removed by filtration and the solution was lyophilized. The lyophilized oligonucleotide (830 mg) was dissolved in a solution of NaCl (2M in 10 mM NaOH, 400 ml), water (600 ml) was added and the pH was adjusted to 7-8 with an aqueous solution of HCl (1M). The oligonucleotide containing solution was purified by desalting as described in the previous examples and lyophilized to give the final product (545 mg, 127 μmol).

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A process for preparing an oligonucleotide consisting of 6 to 16 contiguous nucleotide units, said process comprising the steps of:
(i) preparing a crude mixture comprising an oligonucleotide consisting of 6 to 16 contiguous nucleotide units;
(ii) subjecting the mixture formed in step (i) to a desalting step;
wherein the process does not comprise a chromatographic purification step.

2. A process according to claim 1 wherein step (ii) comprises subjecting the mixture to diafiltration.

3. A process according to claim 1 wherein the mixture formed in step (i) is prepared by the sequential coupling of phosphoroamidite monomers to a nucleotide or oligonucleotide that is covalently bound to a solid support.

4. A process according to claim 1 wherein the oligonucleotide consists of 6 to 12 contiguous nucleotide units, more preferably, 7 to 10 contiguous nucleotide units.

5. A process according to claim 1 wherein the oligonucleotide comprises at least one nucleotide analogue, such as at least one Locked Nucleic Acid (LNA).

6. A process according to claim 1, wherein the oligonucleotide is a LNA gapmer oligonucleotide.

7. A process according to claim 5 wherein all of the nucleotide units are Locked Nucleic Acid (LNA).

8. A process according to claim 1 wherein the nucleotide units are linked by phosphodiester or phosphorothioate linkages, or a mixture thereof.

9. A process according to claim 1 wherein step (ii) is carried out in metal salt solution.

10. A process according to claim 9 wherein the pH is of the metal salt solution is between about 7 and about 8.

11. A process according to claim 2 wherein the diafiltration is carried out in a closed system comprising a reservoir, a pump, a membrane, a detector system and optionally a pressure regulator.

12. A process according to claim 2 wherein the diafiltration flow rate is from about 50 to about 2000 ml/min, more preferably from about 200 to about 400 ml/min.

13. A process according to claim 2 wherein the pressure over the membrane is adjusted to between about 1 to about 3.5 bar, more preferably to between about 2 to about 3 bar.

14. A process according to claim 2 wherein the product formed in step (ii) is subjected to lyophilization.

15. A process for purifying an oligonucleotide consisting of 6 to 16 contiguous nucleotide units, said process comprising subjecting the oligonucleotide to diafiltration, and wherein the process does not comprise a chromatographic purification step.
